(19) United States
(12) Patent Application Publication
(10) Pub. No.: US 2003/0055007 A1
(43) Pub. Date: Mar. 20, 2003

(54) Antimicrobial agents

(76) Inventor: Kazuo Sakuma, Hokkaidou (JP)

Correspondence Address: Kazuo Sakuma, 2119-1, Kaminayoro, Shimokawa-chou, Kamikawa-gun, Hokkaidou, 098-1216, JP

(21) Appl. No.: 09/826,511

(22) Filed: Aug. 2, 2001

(51) Int. Cl. A61K 31/70; A61K 31/075; A01N 31/14

(52) U.S. Cl. 514/22; 514/721; 514/699

(57) ABSTRACT

Purposes

To confirm the efficacy from the chemical substances found in industrial products, which can be supplied with relative stability and continuance, do not produce many side effects, prove that they attain desirable antimicrobial activity and to then make them accepted antimicrobial agents.

Composition

Antimicrobial agents comprising of one or more active constituents having antimicrobial activity, which are selected from guaiacol, lignin sulfonic acid, 2,6-dimethoxyphenol, 3,5-dimethoxyphenol, lignosulfonic acid sodium salt, lignosulfonic acid sodium salt acetate, lignin organosolv propionate, 4-benzyloxyguaiacylglycerol-β-guaiacylether and syringaldehyde.

Effectiveness

Differing from natural products and being antimicrobial agents comprising of industrial products, they are stable from a quantitative and quality point of view, safe and inexpensive and antiviral as well as antibacterial activity can be attained.

### BACKGROUND OF THE INVENTION

1. Field of the Invention

The present invention relates to antimicrobial agents. More particularly, it relates to antimicrobial agents which have inhibitory effects against viruses such as AIDS virus and influenza virus and various kinds of bacteria, wherein the active constituents thereof are guaiacol, lignin family, and related chemical substances.

2. Background Art

In recent years, there has been a growing interest in health food such as dietary fiber and polyphenol. Originally these were chemical substances included in materials available with relative ease and many of them have been traditionally consumed unwittingly. For example, Polyphenol contained in chocolates, has been touted recently by media, is derived from cocoa beans harvested from cacao trees, and it has long been eaten by people unwillingly before it become widely known. The same can be said about polyphenol contained in wine and dietary fiber contained in alimentary yam paste and burdock. Likewise, there are a myriad of chemical substances that are common, affordable, easily obtained and reasonably priced but chemical and biological activities are relatively unknown or they are used for a different purpose. The inventor thought that among chemical substances having unexpected antiviral activity or antibacterial activity might be among them and identification of such substances would offer many benefits and advantages to mankind by ensuring a stable and continuous supply of inexpensive and powerful substances with medicinal properties. It provided an opportunity for me to embark on this invention.

Besides, in the United States about 75% of antibiotics has been used as remedy against influenza for a year. It is said that most of influenza arise from virus infection and antibiotics to destroy bacteria are not effective in treating viruses. By such antibiotic abuse, drug-resistant strains are increasing. In order to prevent such antibiotic abuse, this invention was also undertaken.

In recent years, diseases such as AIDS and influenza are spreading over a wide area and afflicting humanity. In response to this, research scientists have been making every effort to investigate natural substances, but acceptable medicinal properties have not yet been identified. The same can be said for bacteria. In particular, new drugs produced synthetically exert their powerful effects, but they also cause strong side effects. They often bring about an undesirable result where the pathogenic microorganisms are inhibited but human body is exhausted. In addition to synthetic materials, certain natural products are known to have anticancer qualities, antiviral qualities and antibacterial qualities that have been utilized in folk medicine.

However these natural products, which are recognized to be effective against said diseases, have a problem in terms of widespread use in society because of the unstable supply at high volume and high quality.

### SUMMARY OF THE INVENTION

Accordingly, the present invention was undertaken in order to examine many chemical substances that enable us to guarantee a stable and a relatively continuous supply with few side effects and desirable antimicrobial activities. The invention also enables us to establish the effectiveness of the substances and to make them antimicrobial agents.

To solve this problem, at first compounds having a benzene ring were selected extensively as objects of the analysis. Among those chosen were a group of phenol, furthermore chemical substances having hydroxyl groups and methoxy groups, particularly guaiacol and a group of lignin and related chemical substances were targeted in the present invention, whereby the said antiviral and antibacterial activities thereof were evaluated and confirmed. Two varieties of AIDS virus and three kinds of influenza virus were targeted for virus strains and E.coli O157, K.pneumoniae, S.Enteritidis, P.aerginos, S.aureus, B.subtilis, were targeted for bacteria strains. In the invention, the number of chemical substances chosen for the initial test greatly exceeded 100 substances. Tests were particularly run using a great number of the group of lignin, whereby various facts were found. For instance, substances put in the same category as ‘lignin’ were found to have peculiar and uniform properties. With respect to efficacy, certain substances are effective for the AIDS virus but ineffective for the influenza virus, or certain substances are effective for the virus but ineffective on the bacteria, or to the contrary, certain substances are effective on the bacteria but ineffective for the AIDS virus; with respect to the same AIDS virus, inhibitory effects were found on the 3rd day of the cultures but were not found on the 6th day of the culture when the concentrations level of the virus increased. (without day 6 activity) (More detailed information is provided later.). This invention was used on each substance by examining its properties, measuring its molecular weight and analyzing its reconstitution. Then the properties of each substance were confirmed and an evaluation test was run for the effectiveness of each substance, furthermore even if the chemical substances are put in the same category, the activity determined if it was effective or not (i.e. lignin family).

The chemical substances disclosed in the present invention which have been confirmed to be effective in view of the results of various tasks, experiments, research and discussions are 13 types of chemical substance (hereinafter referred to as samples 1-13) and related substances (given by formula 1-13). More detailed information is provided later.

### BRIEF DESCRIPTION OF DRAWINGS

FIG. 1 is a graphical representation to illustrate the molecular weight distribution of reagents used to measure the molecular weights in this invention.

FIG. 2 is a graphical representation to illustrate the molecular weight distribution of lignin sulfonic acid that is one of the antimicrobial agents and is disclosed in this invention.

FIG. 3 is a graphical representation to illustrate the molecular weight distribution of lignosulfonic acid sodium salt concerning this invention.

FIG. 4 is a graphical representation to illustrate the molecular weight distribution of lignosulfonic acid sodium salt acetate concerning this invention

FIG. 5 is a graphical representation to illustrate the molecular weight distribution of lignin organosolv propionate concerning this invention.

FIG. 6 is a graphical representation to illustrate the molecular weight distribution of humic acid examined in relation to this invention.

### DETAILED DESCRIPTION

1. Description of Samples

The samples disclosed in the present invention are the following 13 substances.

(1) Guaiacol This substance is called guaiacol. In addition to that, it is also called hydroxy-2-methoxybenzene, 2-methoxyphenol, O-methoxyphenol, O-hydroxyanisole, 2-hydroxyanisole, methyl-catechol. The structure of this substance is represented by the following formula.

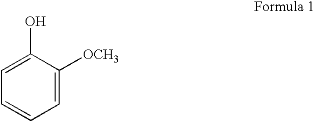

Guaiacol is a mass—manufactured industrial material which is generally useful as an analytical reagent, antiseptic and has other purposes as well. Guaiacol employed in the present invention is a chemical substance with a purity of 98% that is produced and sold by Aldrich Chemical Co., Inc. located in 1001 West St. Paul Milwaukee, Wis. 53233 USA. It is a colorless or faint yellow liquid. It has a boiling point of 205° C. and a melting point of 17-29° C., a flashing point of 82° C. and a specific gravity of 1.129. In contrast to the conventional use stated above, this substance unexpectedly had anti-virus activity (especially against anti-AIDS virus) and anti-influenza virus activity. However, it is surprising that the present investigation revealed that this substance had these benefits and furthermore multi-purpose antimicrobial activity including antibacterial activity. In addition, when guaiacol was screened, guaiacol-4-sulfonate potassium salt 0.5-hydrate, that has a substantially similar chemical structure to guaiacol, was selected as objects of the analysis. Guaiacol-4-sulfonate potassium salt 0.5-hydrate is a substance which is made soluble in water by adding potassium to guaiacol. However, the antiviral activity test revealed that this analogue to guaiacol (guaiacol-4-sulfonate potassium salt 0.5-hydrate) caused cell damage and as a consequence measurement was made impossible. Due to only a minor difference in constituents between the analogs, the result may change greatly. Therefore, we must be careful that our knowledge does not lead to facile reasoning. Guaiacol can be also withdrawn from natural Guaiacum sanctum. The tree trunk of guaiacol is clipped in lengths of about one meter and holes are drilled lengthwise, then the component is eluted by heating over a flame or extracted by boiling the chip. Sap from the tree can also be obtained by scratching the tree. In any case, “guaiac resin” taken from the Guaiac tree is used in industrial materials such as food oxidants, oxidizing agents, oxidase reagents, analytical reagents and various other products. Guaiac resin has been examined for anti-AIDS virus activity in the present invention, together with guaiacol. The substance provided good results that were equal or superior to guaiacol. A detailed outline of the results is given in the description of the test examples using guaiacol described later in this paper. (Refer to [sample 1] in “Complete inhibitory activity against AIDS virus [1]”)

(2) Lignin sulfonic acid The structure of this substance is represented by the following formula.

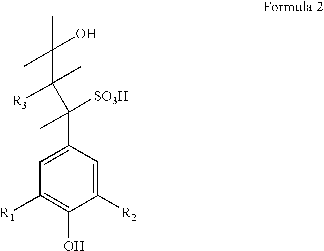

(In which R₁ mostly represents OCH₃, R₂ represents H or other lignin unit and R₃ represents other lignin unit)

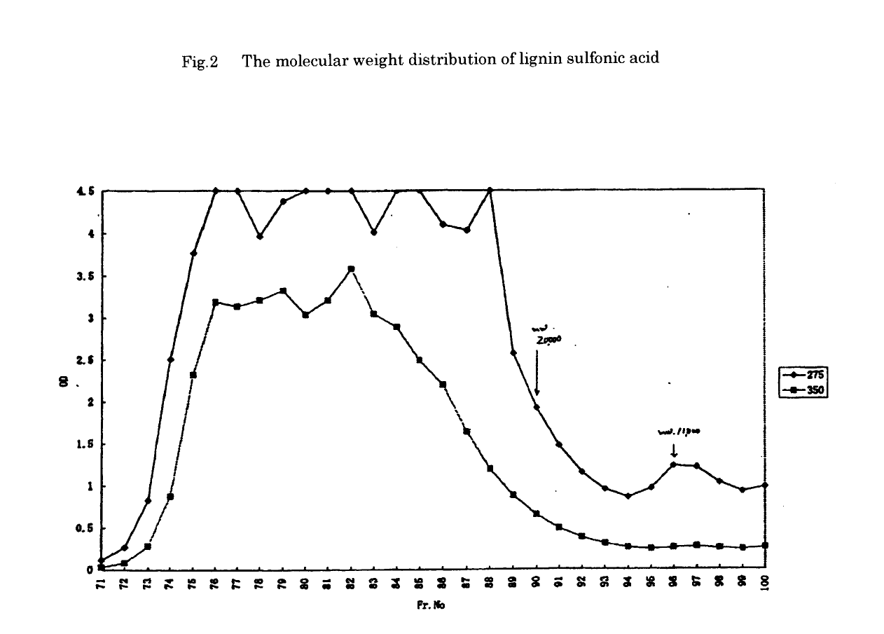

Lignin sulfonic acid is a chemical product produced and sold by Kanto Kagaku Corporation located in 3-2-8, Honcho, Nihonbashi, Tokyo. It is a black powder with a light fragrance of vanilla. The graph in FIG. 2 shows the molecular weight distribution of this substance measured using the method described later. Most of the molecular weights range from 20,000 to 45,000. The present invention revealed the antiviral (against AIDS virus and influenza virus) and antibacterial activity in this substance. Lignin alkali (produced by Aldrich Chemical Co. Inc.) was initially selected from a group of lignin as the subject for research, and an anti-AIDS virus test was conducted using the 3-day and 6-day culture method described later. Compared with the chemical structure of lignin sulfonic acid (formula 2), in the chemical structure of lignin alkali, the H molecule (or other lignin unit)is replaced by SO₃H and R₁ is replaced by R₂. The lignin alkali is closely related to lignin sulfonic acid. So, ordinarily, lignin alkali would be expected to have an equal efficacy compared to lignin sulfonic acid. However, contrary to expectation, the test results of lignin alkali showed no activity on the 6th day. (More detailed information regarding the meaning and significance of day 6 activity is provided later. Refer to “Complete inhibitory activity against AIDS virus [Table2]”). It also helps us to understand that the effectiveness of each substance cannot be determined precisely, using basic “common knowledge” such as presumptions and expectations. It is then understood that the real truth must be obtained using practical tests, other considerations and confirmations.

(3) 2,6-dimethoxyphenol The structure of this substance is represented by the following formula.

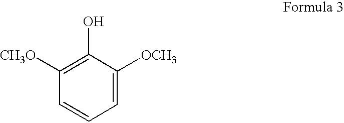

2,6-dimethoxyphenol is a liquid substance sold by Wako Pure Chemical Industries, Ltd.

(Doushu-chou,Chuou-ku,Osaka-shi, Osaka) It is a brown liquid, smelling strongly like oxygenated water. The anti-AIDS virus activity and antibacterial activity of 2,6-dimethoxyphenol were found to be comparable to those of guaiacol.

(4) 3,5-dimethoxyphenol The structure of this substance is represented by the following formula.

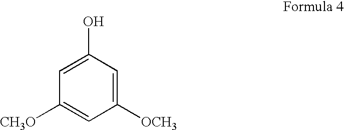

3,5-dimethoxyphenol is a substance produced by Aldrich Chemical Co, Inc. in USA described above. It is a eggshell white crystalline powder with a purity of 99%. It has a boiling point of 172-175° C., a melting point of 45-47° C. and a flashing point of 78° C. It causes irritation to eyes, skin, respiratory tract, etc. This substance was proven to have multi-purpose effects such as anti-AIDS virus and anti-influenza virus activity and antibacterial activity.

(5) Lignosulfonic acid sodium salt The structure unit of this substance (also referred to as lignosulfonic acid sodium) is represented by the following formula.

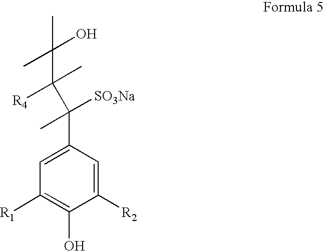

(In which R₁ mostly represents H or other lignin unit, R₂ mostly represents OCH₃ and R₃ represents other lignin unit.)

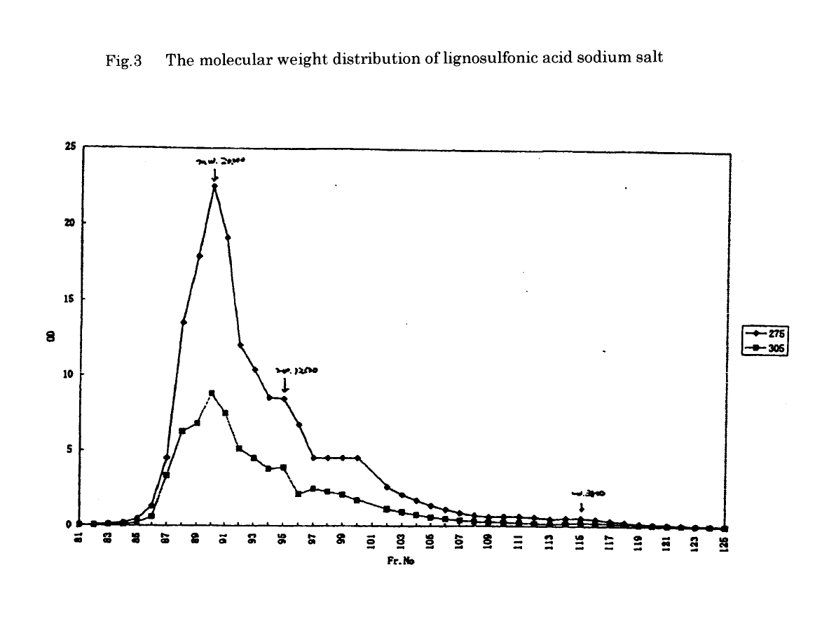

Lignosulfonic acid sodium salt is a substance produced and sold by Aldrich Chemical Co., Inc. in USA mentioned above. It is a polymeric sulfonated lignin material isolated from a commercial pulp mill using predominantly Norway Spruce as raw material. The isolation involves ion exchange (from calcium to sodium) and ultrafiltration. The branched macromolcular structure of lignosulfonic acid sodium salt contains sulfonate groups (degree of sulfonation, 0.46 per phenylpropane repeat unit corresponding to a sulfer content of 6.7%, Na content of 5.5%). It contains primary and secondary aliphatic as well as phenolic OH groups; and it is linked to neighboring phenylpropane repeat units via C—O—C as well as C—C bonds. The methoxyl is ca 10.8%. The elemental analysis shows that it contains 46.17% carbon and 4.70%hydrogen. It is a free-flowing, non-toxic brown powder with a bulk density of ca 0.5 g/cm₃. It is soluble in water. It has a smell resembling ‘Jintan’. Since lignosulfonic acid sodium salt is predominantly used in industrial materials, such as anionic surface-active agents or additives to phenol-formaldehyde resin, the said antiviral and antibacterial activities of this substance, which is the subject of the present invention, were entirely unknown. However it is marvelous that the investigation revealed the excellent anti-AIDS virus and anti-influenza virus activity and a certain degree of antibacterial activity in this substance. The graph in FIG. 3 shows the molecular weight distribution of this substance measured using the method described later. The most abundant molecular weight is 20,000. Molecules with molecular weights of 12,500 and 3,800 were also present.

(6) Lignosulfonic acid sodium salt acetate The structure unit of this substance is represented by the following formula.

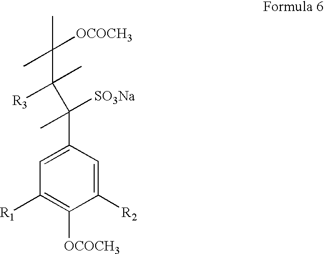

(In which R mostly represents H or other unit, R₂ represents mostly OCH₃ and R₃ represents other unit.)

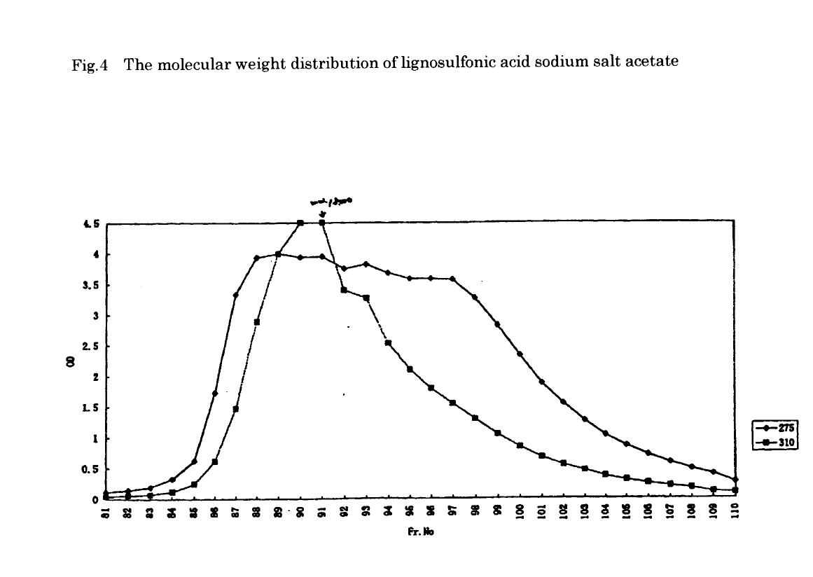

This substance is also produced and sold by Aldrich Chemical Co., Inc. in USA stated above. It is a polymeric derivative of lignosulfonic acid sodium salt. It was prepared in homogenous phase solution using acetic anhydride as reagent. It contains aliphatic and aromatic acetoxy groups. It has ca 0.46 sulfonate groups per phenylpropane repeat unit; and it is linked to neighboring phenylpropane repeat unit via C—C and C—O—C bonds. The elemental analysis shows that it contains 47.16% carbon, 4.72% hydrogen and 3.77% nitrogen. ICP analysis shows that it contains 5.4% Sand 3.1% Na. The predominance of aliphatic over phenolic OH groups was testified. As shown in the graph in FIG. 4, the molecular weight was 18,000, according to the measurement described later. It is a free-flowing, non-toxic yellow brown powder, smelling the faint scent of acetate. It is soluble in water at any pH and insoluble in most organic solvents. Generally it is utilized in industrial materials such as a hydroxyl-free, water soluble material that may be transformed into organic solvent soluble derivatives through appropriate chemical modification. Needless to say, the use as a medical agent that possesses the said diverse qualities was entirely unexpected. However, the present invention clarified the diverse effects of the substance such as strong anti-AIDS virus and anti-influenza virus activities and a certain degree of antibacterial activity, as is so in the case of (5) lignosulfonic acid sodium salt.

(7) Lignin organosolv The structure unit of this substance is represented by the following formula.

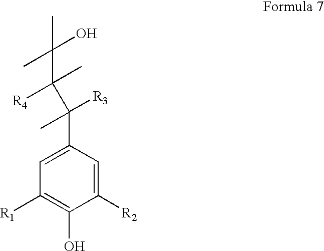

(In which R₁ mostly represents OH₃ or H or other unit, R₂ mostly represents OCH₃, R₃represents OH or other unit and R₄ represents other unit.)

Lignin organosolv is also a chemical substance produced and sold by Aldrich Chemical Co., Inc. It is a polymeric lignin material isolated from a commercial pulpmill using mixed hardwood (mixture of 50% maple, 35% birch, and 15% poplar) as raw material. It contains primary and secondary aliphatic as well as phenolic OH groups. It has an elemental composition of 66.5% carbon, 6.1% hydrogen, 18.9% OCH₃, <0.5% sugars and <1% ash. It is a free-flowing, non-toxic powder with a faint alcoholic odor. It is soluble in aqueous alkali and in selected organic solvents. Generally it is utilized in industrial materials useful for the addition to a phenol formaldehyde resin. However, uses other than that were unknown. The present invention revealed a certain degree of anti-AIDS virus activity in this substance.

(8) Lignin hydrolytic The structure unit of this substance is represented by the following formula.

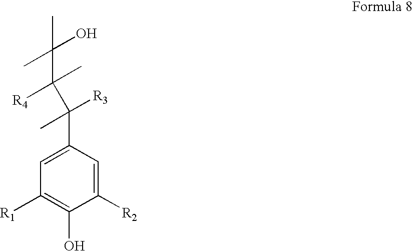

(In which R₁ represents OCH₃ or H or other unit, R₂ represents H or OCH₃, R₃ mostly represents other unit and R₄ represents mostly other unit.)

Lignin hydrolytic is also a chemical substance produced and sold by Aldrich Chemical Co., Inc. It is a polymeric autohydrolysis lignin material isolated from a commercial hydrolysis pilot plant using predominantly sugar cane bagasse as raw material. The branched macromolecular structure of it contains primary and secondary aliphatic OH groups as well as more superior phenolic OH groups. It has a methoxyl content in the range of 9-11%. It is linked to neighboring phenylpropane repeat units via C—C and C—O—C bonds. It is a free-flowing, non-toxic brown powder. It is soluble in aqueous alkali and in selected organic solvent mixtures (i.e. methanol or ethanol plus acetone, methylene chloride, chloroform, or benzene). Generally it can be added to a phenol formaldehyde resin. The present invention revealed a certain degree of anti-AIDS virus activity in this substance.

(9) Lignin organosolv acetate The structure unit of this substance is represented by the following formula.

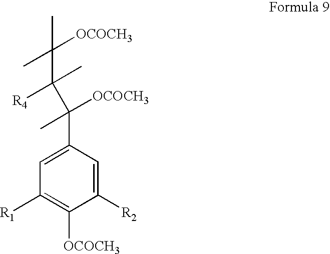

(In which R₁ represents OCH₃ or H or other unit, R₂ represents OCH₃ and R₃ mostly represents other unit.)

Lignin organosolv acetate is also a chemical substance produced and sold by Aldrich Chemical Co., Inc. It is a polymer derivative of lignin organosolv stated above. The branched macromolecular structure of it contains primary and secondary aliphatic as well as aromatic acetoxy groups. It has a small amount of CO and/or COOH groups. It is a free-flowing, non-toxic light brown powder and is soluble in most organic solvents and insoluble in water. This substance is used as a thermoplastic material that may be added to other polymeric materials in the melt as viscosity modifier, colorant or for other purposes. The present invention revealed a certain degree of anti-AIDS virus activity in this substance.

(10) Lignin hydrolytic hydroxymethyl The structure unit of this substance is represented by the following formula.

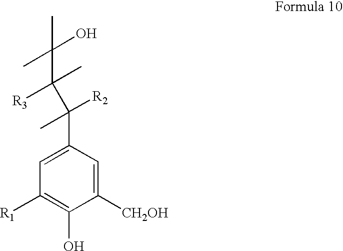

(In which R₁ mostly represents OCH₃ or other unit, R₂ represents OH or other unit and R₃ mostly represents other unit.)

Lignin hydrolytic hydroxymethyl is also a chemical substance produced by Aldrich Chemical Co. Inc. It is a polymeric derivative of lignin hydrolytic (8) stated above. This is prepared by treating lignin in homogeneous solution (aqueous alkali) with formaldehyde. The branched macromolecular structure of it contains primary and secondary aliphatic as well as phenolic OH groups. It has a small amount of carboxy functionality. It is a free-flowing, non-toxic black powder and is soluble in aqueous alkali and selected organic solvent mixtures. It is utilized in industrial materials useful for the addition to a phenol formaldehyde resin. The present invention revealed a certain degree of anti-AIDS virus activity in this substance.

(11) Lignin organosolv propionate The structure unit of this substance is represented by the following formula.

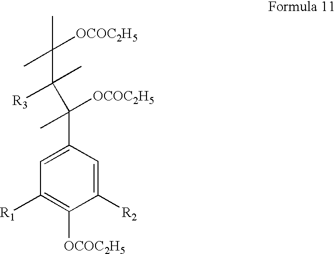

(In which R₁ mostly represents H or OCH₃ or other unit, R₂ mostly represents OCH₃ and R₃ mostly represents other unit.)

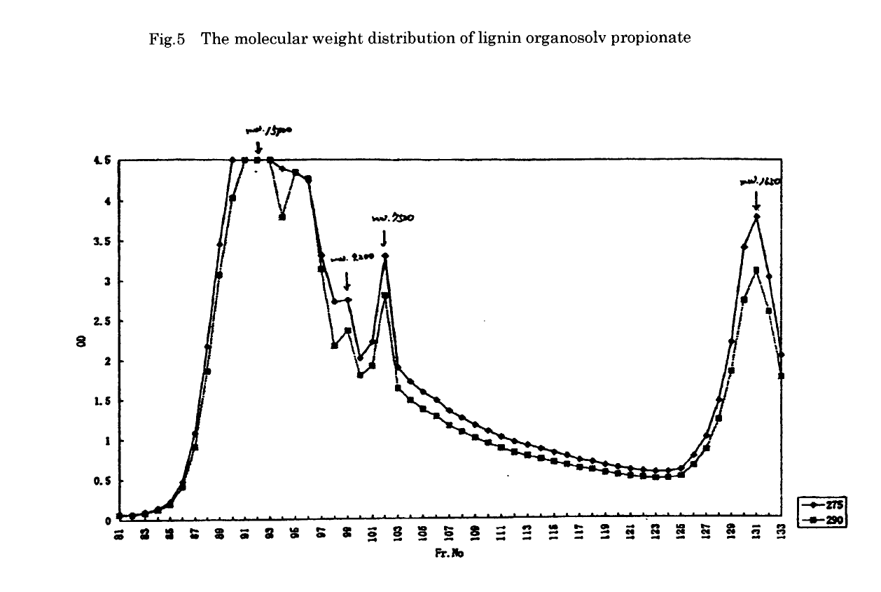

Lignin organosolv propionate is also a chemical substance produced and sold by Aldrich Chemical Co., Inc. The molecular weight distribution is shown in the graph in FIG. 5. The most-abundant molecular weight is 15,000. Molecules with molecular weights of 9200, 7500 and 1650 were present. This substance is a polymer derivative of lignin organosolv (7) stated above. It is prepared by homogenenous phase reaction of lignin in propionic acid, using propionic anhydride as reagent and sodium propionate as catalyst. Prior to isolation, partially bleached with H₂O₂, it became a brown powder. The elemental analysis shows that it contains 57.39% carbon and 5.58% hydrogen. It is a free-flowing, non-toxic powder. It is utilized as a well-soluble thermoplastic lignin derivative useful for the addition to polymeric materials and plastics as colorant, viscosity modifier, and other purposes. The present invention revealed the anti-AIDS virus activity and antibacterial activity in this substance.

(12) 4 benzyloxyguaiacylglycerol β-guaiacylether The structure of this substance is represented by the following formula.

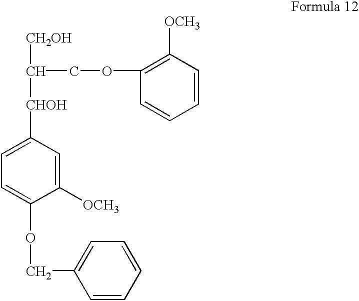

4-benzyloxyguaiacylglycerol-β-guaiacylether is a chemical substance provided by Hokkaido University. It is a whitish powder. The present invention revealed a certain degree of anti-AIDS virus activity and anti-influenza virus activity in this substance. SOS which is related to this substance is described later in the paper.

(13) Syringaldehyde The chemical structure of this substance is represented by the following formula.

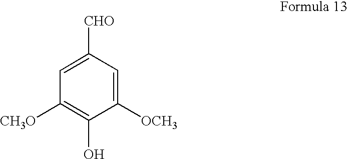

Syringaldehyde is a chemical substance produced and sold by Aldrich Chemical Co., Inc. stated above. But the present invention revealed that the substance showed considerably strong anti-AIDS virus activity as well as severe cell damage. When applied, it is preferred to lower the cell damage by using it together with other chemical substances, etc.

2. Results for Molecular Weight Determination

Among the said variety of lignin family, the determination of molecular weights was made for the following 4 samples and related humic acid, which is described hereunder (see “proximate analysis of lignins, etc.” [Table 1]).

A. Reagents, instruments, etc.

(1) Lignin Specimens

1. Lignin sulfonic acid

2. Lignosulfonic acid sodium salt

3. Lignosulfonic acid sodium salt acetate

4. Lignin organosolv propionate

(5. Humic acid)

(2) Reagents

1. Blue Dextran 200 (Pharmacia Fine Chemicals, Inc.)

2. Polyethylene glycol 400, 1000, 4000, 6000, 20000 (Wakou first class grade) (abbreviated to PEG)

3. methanol (Wakou special grade chemicals)

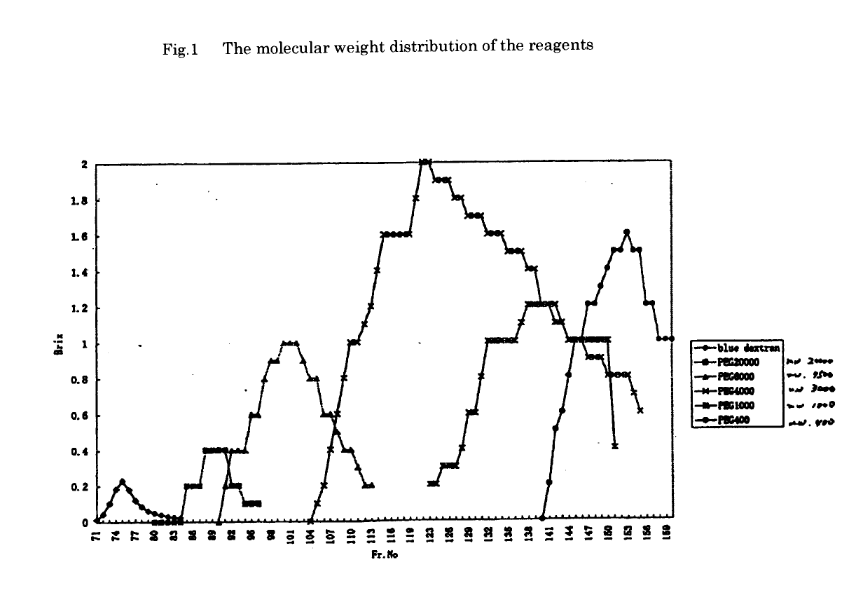

(The molecular weight distribution of the reagents land 2 is shown in the graph in FIG. 1)

(3) Equipments

1. Fraction collector (ADVANTEC SF-2120)

operating condition; simple mode

waiting time; 0 minutes

fraction 85 drop/tube (molecular weight marker)

120 drop/tube (lignin) (each 2.5 ml tube)

2. Spectrophotometer (BECRUN DU 7400)

3. Brix scale (ATAGO HAND REFRACTOMETER N-1E)

(4) Column

gel filtration medium TOYOPARL HW50F (Tosoh Corp.)

a column equipped with a tap (2.5×120 cm, glassware)

B. Test procedure

(1) Preparation of a Column

The column was fixed upright onto the stand. After washing the gel filtration medium in deionized water, the medium was loaded into the column, using a routine method (falling). The upper solvent tank was connected to the upper end of the column, and the column was washed with deionized water in a volume three times that of the gel filtration medium. The lower tap of the column and the fraction collector were connected.

(2) Elution of Molecular Weight Markers

Molecular weight markers (Blue Dextran) and Five different PEGs (polyethyleneglycols), used as molecular weight markers, were dissolved in deionized water at concentrations of 5-10%. The deionized water within the column was allowed to fall to the level of the top of the gel filtration medium, and the lower tap was closed. Then, 5 ml of the marker solution was added, taking care to avoid disturbing the surface of the gel filtration medium. The lower tap was opened, and the solution to be added was dropped onto the surface of the gel filtration medium. Elution in deionized water was then started. The eluate was fractionated in the fraction collector. Each fraction was checked for markers, using a spectrophotometer (Blue Dextran; wavelength=595 nm) and a Brix meter (PEG). An elution curve and a molecular weight standard curve were drawn, with the fraction containing the largest proportion of the marker being regarded as the location of elution of each marker.

(3) Elution of Lignins

The columns were washed with 50% (v/v) methanol/deionized water in a volume twice that of the gel filtration medium and then equilibrated. 20 g of lignins dissolved in 50% methanol solutions were added to the columns respectively. Each eluate was fractionated by 50% methanol, according to the same procedure as above. Eluted lignin in each fraction was measured by spectrophotometry The maximum absorption wavelength of each lignin solution was measure

| | |
|---|---|
| lignin, | wavelength (nm) |

| | | |
|---|---|---|
| 1.Lignin sulfonic acid | 275 | 350 |
| 2.Lignosulfonic acid sodium salt | 275 | 305 |
| 3.Lignosulfonic acid sodium salt acetate | 275 | 310 |
| 4.Lignin organosolv propionate | 275 | 290 |

By drawing elution curves of each lignin, the fractions containing the largest proportion were measured. The molecular weights were estimated through the molecular weight curves drawn in advance (each lignin 1-4 correspond to graphs in FIG. 2-FIG. 5)

[3] Results

1. Lignin Sulfonic Acid

Most-abundant molecular weights range from 20000 to 45000 Molecules with molecular weights of 1,1000 were present.

2. Lignosulfonic Acid Sodium Salt

The most-abundant molecular weight is 20000. Molecules with molecular weights of 12,500 and 3,800 were present.

3. Lignosulfonic Acid Sodium Salt Acetate

The molecular weight was 18000.

4. Lignin Organosolv Propionate

The most-abundant molecular weight is 15000. Molecules with molecular weights of 9200, 7500, 1650 were present.

5. The Molecular Weight of Humic Acid

The molecular weight of humic acid was estimated in the same way as the analysis of the molecular weights of lignins. In this regard, 30 mg of humic acid dissolved in 5 ml of 0.1 N sodium hydroxide solution and eluted with 0.1 N sodium hydroxide solution was used.

Fraction collector: condition, simple mode

75 drop/Fr. (25 ml) sodium hydroxide solution

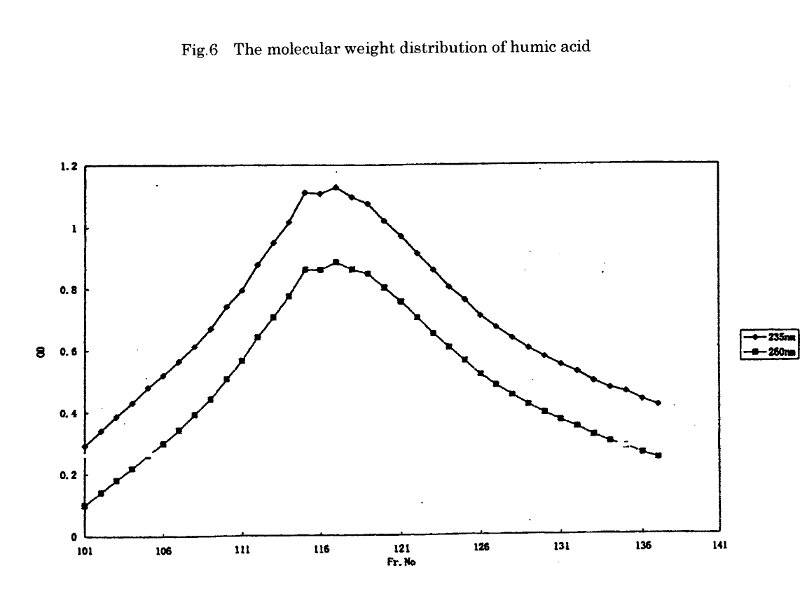

As shown in the graph in FIG. 6, elution peak was present in Fr. No.117 and the molecular weight of humic acid was estimated to be approximately 3500 through the analytical curve of the molecular weight.

2. Proximate Analysis

The proximate analysis performed for lignins, other natural lignins employed in the present invention and humic acid, which is related to them, is described hereunder. The following samples were used.

Samples

1) Lignin sulfonic acid (sample 2)

2) Lignosulfonic acid sodium salt (sample 5)

3) Lignosulfonic acid sodium salt acetate (sample 6)

4) Lignin organosolv propionate (sample 11)

A Lignin sulfate fraction of mushrooms

B Lignin hydrochloride fraction of mushrooms

C Humic acid

A. Technique

Each sample (excepting C which is described later) accurately measured to be 200 mg/ml was dissolved in deionized water. After each sample was stirred thoroughly and centrifuged at 3,000 rpm for 15 minutes, the supernatant solutions thereof were taken and subjected to the following test.

Employed instruments: spectrophotometer; Shimazu spectrophotometer UV 1200

pH meter; Toua Digital pH meter-50

B. Test

(1) OD 500 nm and pH

1% (W/V) aqueous solutions of each sample were prepared, followed by measurement of absorbance at 500 nm and pH, using a routine method.

(2) Protein

The amount of protein contained in each sample solution was assayed by the Bradford method. The sample solution, 20 μl, was placed into a 1.5 ml test tube and was combined with 1 ml of Bradford solution. The mixture was left standing at room temperature for 5 minutes, followed by measurement of absorbance at 595 nm. As control solution, Bradford solution added 20 μl deionized water was used. The sample solution was diluted appropriately before measurement, to avoid influences from impurities contained in the sample. The amount of protein contained in the sample solution was calculated, referring to a calibration curve created using bovine serum albumin as a reference. The protein content per 1 g sample was obtained.

(3) Glucose

Using glucose C-II Test Wako (Wako Pure Chemical Industries, Ltd.), the amount of glucose contained in each sample solution was measured. The reagent used for this measurement was an enzymatic reagent with high specificity. The sample solution, 20 μl, was placed into a test tube and was combined with 3.0 ml color-producing reagent. After warmed to 37° C. for 5 minutes, the absorbance of each solution was measured at 505 nm. sample solution added 3.0 ml of deionized water was used for the control group to eliminate the color effect. Calibration curves were produced by simultaneous reaction to the glucose standard solution The amount of glucose contained in the sample solution was calculated, referring to the calibration curve and converted into the amount per 1 g sample

(4) All Saccharides

Using a phenol-sulfuric acid method, the total amount of saccharide in each sample was measured. The sample solution, 20 μl was placed into a test tube and was combined with 5% phenol solution. 1 ml of concentrated sulfuric acid was added dropwise to the test tube. Stirred rapidly and left standing at room temperature for f20 minutes, the absorbance was determined at 490 nm. Distilled water, instead of the sample solution, was used for the control group. Calibration curves were produced by simultaneous reaction to the glucose standard solution. The total amount of saccharides contained in the sample solution was calculated as the amount of glucose, referring to the calibration curve and converted into the amount per 1 g sample. The analysis test results are given in the following Table 1.

TABLE 1
Proximate analysis of lignins, etc

| | 1% solution | | protein | glucose | all saccharides |
|---|---|---|---|---|---|
| sample | 500 nmOD | pH | (mg/g) | (mg/g) | (mg/g) |
| 1 Sample 2 | 1.405 (×10) | 8.87 | 61.36 | 0.0 | 357.1 |
| 2 Sample 5 | 0.579 | 8.79 | 19.19 | 4.56 | 239.1 |
| 3 Sample 6 | 0.347 | 4.00 | 24.68 | 0.27 | 224.8 |
| 4 Sample 11 | 0.300 | 4.86 | 4.38 | 0.44 | 0.399 |
| A Sulfate | 0.024 | 3.24 | (0.36) | 0.35 | 0.323 |
| fraction | | | | | |
| B Hydrochloric | 0.014 | 3.12 | (0.43) | 0.28 | 0.318 |
| fraction | | | | | |
| C Humic acid | 0.030 | 5.36 | 1.30 | 0.18 | 0.141 |

As described above, in the present invention, more than 100 kinds of substances which are mostly likely to be candidate substances for antimicrobial qualities were chosen and have been researched in earnest. The said 13 types of chemical substances were selected carefully among them and proven to be effective or partly effective. The tests were run regarding \mathring{1} complete inhibitory activity against AIDS virus, \mathring{2} complete inhibitory activity against influenza virus, and \mathring{3} antibacterial activity which will be described later. The information regarding the complete inhibition against of AIDS virus is described hereunder.

3. Complete Inhibitory Activity Against AIDS Virus

Sample solutions were prepared by dissolving the said samples (more than 100 kinds of chemical substances stated above) in water. The initial concentration of aqueous solutions was determined at 1 mg/l ml (Powdered sample per 1 ml aqueous solution 1 mg=1000 μg).2 varieties of AIDS virus (HIV-1, HIV-2) and suspension of MT-4 cells are prepared independently and a 12-well microplate was used. The sample, as aqueous solution, was added to the first well at a concentration of 1,000 μg/ml. The sample was serially diluted by a ratio of 2 in each of the subsequent wells (500 μg/ml for the second well, 250 μg/ml for the third, 125 μg/ml for the fourth, 62.5 μg/ml for the fifth, and 0.49 μg/ml for the twelfth). The dilution ratio for the 12th well was 1:2048. The following Table shows the sample concentration (μg/ml) (A) and the dilution ratio (B) for each well.

| well | 1 | 2 | 3 | 4 | 5 | 6 | 7 | 8 | 9 | 10 | 11 | 12 |
|---|---|---|---|---|---|---|---|---|---|---|---|---|
| A | 1000 | 500 | 250 | 125 | 62.5 | 31.3 | 15.6 | 7.81 | 3.91 | 1.95 | 0.97 | 0.49 |
| B | 1 | 2 | 4 | 8 | 16 | 32 | 64 | 128 | 256 | 512 | 1024 | 2048 |

100 μl of each sample solution at the concentration described above and 100 μl of each suspension of AIDS virus were placed in a microplate well; thus, AIDS virus in sample solution was cultured. On the 3rd and 6th day of the cultures, complete inhibitory effects against the growth of AIDS virus were determined for each well. “Complete inhibitory effects against the growth” indicate that MT-4 cells coexisting with AIDS virus remain in a healthy state, have not been destroyed and have not degenerated through the AIDS virus. The viral concentration level was low, 10 TCD, on the 3rd day of the cultures. But in the control group it rose up to 100 TCD on the 6th day. Therefore, “day 6 activity” in the increasing concentration range that completely suppressed the viral growth is an important criterion for evaluation of the effectiveness of samples. The samples 1-7 which showed remarkable results were selected through the evaluation test and the results are given in Table 2 (samples 1-7 correspond to said formulas 1-7). In Table 2, a circle marked on the well number indicates that AIDS virus growth was inhibited completely (100%) in the well concerned and wells with lower numbers. The number in the parentheses indicate the concentration of the sample in that well (μg/ml). AIDS virus growth was not inhibited completely in wells with higher numbers. (the wells located to the right of the circle-marked well). The mark “*” marked on the well number indicate that cell damage was caused not by the virus but due to the sample compounds.

TABLE 2
Completely inhibitory activity against AIDS virus (1)
(samples 1-7 correspond to said formulas 1-7)

| | | well number | | | | | | | | | | | | |
|---|---|---|---|---|---|---|---|---|---|---|---|---|---|---|
| samples | culture | 1 | 2 | 3 | 4 | 5 | 6 | 7 | 8 | 9 | 10 | 11 | 12 | abbr. |
| 1 | 3-day | | | * | | | ◯ (31.3) | | | | | | | 6T₃ |
| | 6-day | | | * | | ◯ (125) | | | | | | | | 4T₃ |
| 2 | 3-day | | | | * | | | | ◯ (7.81) | | | | | 8T₄ |
| | 6-day | | | | * | | | ◯ (15.6) | | | | | | 7T₄ |
| 3 | 3-day | | | | * | | ◯ (31.3) | | | | | | | 6T₄ |
| | 6-day | | | | | * | ◯ (31.3) | | | | | | | 6T₅ |
| 4 | 3-day | | * | | ◯ (125) | | | | | | | | | 4T₂ |
| | 6-day | | | * | ◯ (125) | | | | | | | | | 4T₃ |
| 5 | 3-day | | | | | | | | | | ◯ | | | 10T₀ |
| | 6-day | | | | | | | | | ◯ | | | | 9T₀ |
| 6 | 3-day | | | | | | | | | ◯ | | | | 9T₀ |
| | 6-day | | | | | | | | | | ◯ | | | 10T₀ |
| 7 | 3-day | | | | | * | | ◯ | | | | | | 7T₅ |
| | 6-day | | | | | * | < | | | | | | | <6T₅ |

sample 1 = guaiacol
sample 2 = lignin sulfonic acid
sample 3 = 2,6-dimethoxyphenol
sample 4 = 3,5 dimethoxyphenol
sample 5 = lignosulfonic acid sodium salt
sample 6 = lignosulfonic acid sodium salt acetate
sample 7 = lignin organosolv

In sample of specimen 1 (guaiacol), the viral growth was completely suppressed up to well 6 (31.3 μg/ml) by 3-day incubation, and up to well 4 (125 μg/ml) by 6-day incubation. Cell damage was observed up to well 3 by both 3- and 6-day incubation (250 μg/ml). The results are given on the right side of Table 2. The abbreviations were as follows: 6T₃ was the viral activity on the 3rd day; 4T₃ was the viral activity on the 6th day (T was abbreviated as Toxicity). An evaluation test for Anti-AIDS virus activity was run for guaiacol (sample 1) and guaiac resin including guaiacol, which is the extract from natural Guaiac tree. Good results were obtained. The viral activity was 3.9 μl/ml on the 3rd day (cell damage, 7.8 μl/ml) (abbreviated as 9T₈) and 3.9 μl/ml on the 6th day (cell damage, 7.8 μl/ml) (abbreviated as 9T₈). Trough the present investigation, guaiac resin appeared to be comparable to guaiacol in terms of anti-viral activity (especially against AIDS virus). Although, guaiac resin is a natural product, it can be readily produced as an industrial product as described above. Therefore, like guaiacol, this is included in the scope of the present invention.

In sample 2 of specimen 2 (lignin sulfonic acid), the viral growth was completely suppressed up to well 8 (7.81 μg/ml) by 3-day incubation, and up to well 7 (15.6 μg/ml) by 6-day incubation. Cell damage was observed up to well 4 by both 3-day and 6-day incubation. 8T₄ in day 3 activity and 7T₄ in day 6 activity indicate that the sample possessed strong inhibitory activity against AIDS virus. In sample 3 of specimen (2,6-dimethoxyphenol), the complete viral inhibitory activities were 6T₄ on the 3rd day, and 6T₅ on the 6th day. It exerted high cytotoxicity. The same can be said of the activity of this sample against influenza virus, this is described later. The activity of this substance in inhibiting the virus itself is not weak, but it injures the cells used for incubation of the virus, thus making it difficult to show higher activity for this substance. However, the fact that 2,6-dimethoxyphenol inhibited the virus growth strongly on the 6th day (up to well 6) was quite unexpected. As a consequence, this substance was well recognized to be an effective antiviral agent.

In sample 4 of specimen (3,5-dimethoxyphenol), the viral growth was completely suppressed up to well 4 at a concentration of 125 μg/ml by 6-day incubation, although its potency was lower than that of sample 3. In 4T₃ of day 6 activity, cell damage occurred in well 3 immediately before the well where the 100% inhibition activity was observed. However, when an appropriate amount of 3,4-dimethoxyphenol (in formula 4, which has methoxy groups in positions 3 and 4) was added to this sample (e.g. at a ratio of 1:1), the concentration of each component was halved, and cell damage was seen only in the first well, without reducing the anti-viral effect. Other substance such as albumin can be used in mixtures as substitutes for 3,4-dimethoxyphenol. This way of reducing the cytotoxic action is also valid for many other samples. The mixture of 2 or 3 agents can thus reduce the cytotoxicity without compromising the anti-viral activity. Now, we refer to the lignin fraction, originating from mushrooms described previously in “proximate analysis of lignins” (Table1), although this does not directly pertain to the object of the present invention. Tests conducted according to the procedure, similar to the ones described above, confirmed that these lignin fractions exert anti-HIV and other antimicrobial activity. It has been shown that the addition of albumin to these fractions at a ratio of 1:1 reduces the cytotoxicity from T₃ to T₁ (These lignin fractions have been identified by the inventor).

In sample 5 of specimen (Lignosulfonic acid sodium salt) and sample 6 of specimen (lignosulfonic acid sodium salt acetate), the complete growth inhibitory effects were remarkable. The results are given in Table 1. 10T₀ means that the viral growth was completely suppressed up to well 10 (1.95 μg/ml) where the concentration level was low, and cytotoxicity was not observed. 9T₀ also means that the viral growth was completely suppressed at low concentration (3.91 μg/ml) and cytotoxicity was not observed. This suggests that sample 5 and sample 6 possess strong anti-AIDS virus activity and they can be made nontoxic Anti-AIDS virus agents, using them in combination with food and drink. Furthermore, high therapeutic efficacy is anticipated in these substances. Both substances were quite effective against influenza virus and are expected to exert considerable anti-bacterial activity. They are thus expected to exert at least 3 different effects (anti-viral, anti-bacterial, and other effects). They are promising as multi-purpose anti-microbial agents useful for diverse purposes and will be used frequently clinically.

When sample 7 of specimen (lignin organosolv) was incubated for 6 days (Table 2), complete inhibition of viral growth was not seen in the 6th well, indicating that the minimum level causing complete inhibition is higher than the concentration in the well 6th well (<). Cell damage was caused in the 5th well (<6T₅). Accordingly, it is not possible to make use of this substance alone as an AIDS virus agent. But the cytotoxicity of this substance can be reduced, through using it as a mixture or concomitantly with some other agents.

Complete inhibitory activity test (100%) was conducted for the samples other than the said substances, using a microplate according to the procedure, similar to the ones described above. (The chemical substances correspond to formula 8-formula 13.)

The results are given in Table. The same abbreviations are used as above.

TABLE 3

| samples | 3-day culture | 6-day culture |
|---|---|---|
| 8 Lignin hydrolytic | <6T₅ | 5T₄ |
| 9 Lignin organosolv acetate | 6T₅ | <6T₅ |
| 10 Lignin hydrolytic hydroxymethyl | <6T₅ | 3T₂ |
| 11 Lignin organosolv propionate | 6T₅ | 5T₄ |
| 12 4-benzyloxyguaiacylglycerol | 5T₃ | 4T₃ |
| βguaiacylether | | |
| 13 Syringaldehyde | 11T₈ | 10T₉ |

For sample 11 of specimen (lignin organosolv propionate), the well exhibiting 100% inhibition was close to the well showing cell damage. When used as a mixture or concomitantly with some other agents which can reduce the cytotoxicity, as mentioned above, it is possible to make use of this substance as a useful anti-HIV agent that has 100% inhibition in the 5th well (62.5 μg/ml). In addition, lignin organosolv propionate is effective as an antibacterial agent as described below, so it can be a multi-purpose antimicrobial agent, as with the said sample 5 and sample 6.

In relation to the test for sample 12 (4-benzyloxyguaiacylglycerol-β-guaiacylether), anti-AIDS virus activity test was conducted for SOS (syringylglycerol-β-syringyl ether), according to the procedure, similar to the ones described above. SOS is said to have a similar lignin model structure as that of sample 12. The inhibitory activity against the virus on the 3rd day was 15.6 μl/ml (cell damage, 31.3 μl/ml) (7T₆), but unfortunately the viral inhibitory activity was not shown on the 6th day of cultures. In 4-benzyloxyguaiacylglycerol-β-guaiacylether, sample 8 of the lignin model, 5T₃ in day 3 activity and 125 μl/ml (4T₃) in day 6 activity were observed, indicating that this sample had sufficient anti-AIDS activity. These results indicated that the simple interpretation that all kinds of lignin have the same effectiveness is erroneous.

The anti-AIDS virus activity of sample 13 (Syringaldehyde) was outstanding in that on the 6th day the viral activity was observed up to well 10 where the concentration level was low (3.91 μg/ml). This substance also showed anti-influenza virus activity. It may exert antibacterial activity when the concentration level increased. Therefore, it is possible to make use of this substance as a multi-purpose antimicrobial agent. It is difficult to recognize the other samples 8, 9, 10 as effective anti-AIDS virus reagents, only by using them independently. as is so in the case of sample 7 (lignin organosolv) Although I supply this information for reference purposes, through a similar cell-level test as above, 0.5 hydrate of guaiacol 4-sulfonate potassium salt was found not to possess effective anti-AIDS virus activity.

The inventors do not intend to limit the mechanism by which anti-AIDS virus activity is exerted by the above-mentioned chemicals to any particular one. However, in view of the results of various experiments and discussions, it is suggested that the chemicals of the invention effectively inhibit protease, i.e., block the adherence of protease (produced by HIV) to the target cells by physically masking the target cells. Likewise, it was estimated that the said chemical substances of the present invention had similar mechanisms for inhibiting influenza virus.

5. An Evaluation Test Regarding Inhibitory Effects Against the Growth of Influenza Virus

Among many chemical substances of the present invention, the said substances, which appeared to have anti-AIDS virus activity, were used in an evaluation test conducted regarding inhibitory effects against the growth of influenza virus; that is, guaiacol (formula 1), lignin sulfonic acid (formula 2), 2,6-dimethoxyphenol (formula 3), 3,5-dimethoxyphenol (formula 4), lignosulfonic acid sodium salt (formula 5), lignosulfonic acid sodium salt acetate (formula 6), lignin organosolv propionate (formula 11), 4-benzyloxyguaiacylglycerol-β-guaiacylether (formula1 2) and syringaldehyde (formula 13)

The next three kinds of viruses were employed.

1) Influenza virus A/Hokkaidou/1/96 (H1N1) (hereinafter referred to as H1N1)

2) Influenza virus A/Hokkaidou/1/97 (H3N2) (hereinafter referred to as H3N2)

3) Influenza virus B/Hokkaidou/1/97 (hereinafter referred to as Type B)

Preparation of MDCK Maintenance Solution

To maintain MDCK cells for inoculating influenza virus, MDCK maintenance solution was prepared.

Composition of MDCK Maintenance Solution

1. Basal medium: Eagle's MEM 88 ml

2. Penicilin/Streptomycin: each 200 units/ml

(One ml of a 20,000 unit/ml solution was added to 100 ml.)

3. Glutamine: 0.03% addition (One ml of 3% solution was added to 100 ml.)

4. Glucose: 0.01% addition (One ml of 1% solution was added to 100 ml.)

5. Bovine Albumin (fraction V): 0.2% addition (2 ml of 10% solution was added to 100 ml.)

6. Vitamin: 4% addition (4 ml of 100-fold diluted solution is added to 100 ml.)

7. pH modification: pH was modified within a range of 7.6 to 7.8 (3 ml of 5% solution was added to 100 ml.)

Each sample (formulas 1-6, 11-14) was diluted stepwise with culture of MDCK cells. Initial concentration of the sample stock solution was determined at 10 mg/ml (10 μl/ml of liquid guaiacol and 2μ1/ml of 2,6-dimethoxyphenol, indicated by * in Table 4). Test tubes were prepared, and 0.2 ml of the sample stock solution and serial dilutions up to 1:128 (in addition to 1:2, 1:4, 1:8, 1:16, 1:32 and 1:64, there were 1:2.6, 1:5.3, 1:10 and 1:100 dilutions) were added to 1 ml culture medium with 0.2 ml of 10 TCID virus solution. The determination was made, referring to the dilution ratios of each sample, to inhibit the virus growth when usually the virus multiplied up to 100 TCID (3-4 days after inoculation) as to the most effective amounts. Table 4 shows their minimum inhibitory concentration (MIC) against influenza virus (in parentheses the same abbreviations are used as above. For example, 3.5T₁ indicates that complete inhibition was exhibited in between test tube No.3 (1:4 dilution) and test tube No.4 (1:8 dilution).

TABLE 4
Minimum inhibitory concentration (MIC)
(The numbers of the samples correspond to
formulas 1-6, 11-13)

| | influenza virus | | |
|---|---|---|---|
| sample | H1N1 | H3N2 | B |
| 1 | 3.8 μl/ml (2.5T₁) | 1.9 μl/ml (3.5T₁) | 3.8 μl/ml (2.5T₁) |
| 2 | 1.9 mg/ml (3.5T₁) | 1.9 mg/ml (3.5T₁) | 2.5 mg (3T₁) |
| *3 | 1.0 μl/ml [symbol] (<2T₁> | as follows | as follows |
| 4 | 5.0 mg/ml (2T₁) | 1.9 mg/ml (3.5T₁) | 3.8 mg/ml (2.5T₁) |
| 5 | 0.16 mg/ml (7T₀) | 0.10 mg/ml (8T₀) | 0.63 mg/ml (5T₀) |
| 6 | 0.16 mg/ml (7T₀) | 0.16 mg/ml (7T₀) | 1 mg/ml (4.5T₀) |
| 11 | 2.5 mg/ml (3T₁) | 2.5 mg/ml (3T₁) | 3.8 mg/ml (2.5T₁) |
| 12 | 8.0 mg/ml (2T₁) | 2.5 mg/ml (3T₁) | 2.5 mg/ml (3T₁) |
| 13 | 3.8 mg/ml (2.5T₁) | 1.9 mg/ml (3.5T₁) | 3.8 mg/ml (2.5T₁) |

In the experiments shown in Table 4, 2,6-dimethoxyphenol was not effective for influenza virus actually, partly due to the initial concentration level (2 μl/ml). However, all the samples excepting 2,6-dimethoxyphenol exerted significant inhibitory effects. Most remarkable here are lignosulfonic acid sodium salt (sample 5) and lignosulfonic acid sodium salt acetate (sample 6). Lignosulfonic acid sodium salt inhibited the growth of influenza virus, H3N2 completely even at very low concentration (1:100 dilution), and exerted 100% inhibitory effects against influenza virus, Type B at low dilution rates in test tube No.5 (1:16 dilution). It was the remarkable result that lignosulfonic acid sodium salt acetate (sample 6) showed the minimum inhibitory level of 7To (against H1N1and H3N2) and 4.5T₀ (against Type B in the 1:10 dilution). Likewise, it was also suggested that guaiacol (sample 1), lignosulfonic acid (sample 2), 3,5-dimethoxyphenol (sample 4), lignin organosolv propionate (sample 11), 4-benzyloxyguaiacylglycerol-β-guaiacylether (sample 12), syringaldehyde (sample 13) might be effective anti-influenza virus agents, by using in combination with food and drink.

Subsequently another evaluation test regarding inhibitory activity against influenza virus was run for the same influenza virus (H1N1, H3N2, Type B) and lignosulfonic acid sodium salt (sample 5) and lignosulfonic acid sodium salt acetate (sample 6). This test differs from the previous one in that the initial concentration of the sample stock solution was determined at 2 mg/ml (1:2 dilution). Strikingly, even at this high concentration level, cell damage was not seen. The results regarding MIC are given in Table 5.

TABLE 5
Evaluation of minimum inhibitory concentration (MIC)
for influenza virus (2) (a high level of concentration)

| | influenza virus | | |
|---|---|---|---|
| sample | H1N1 | H3N2 | B |
| 5 | 83.3 μg/ml | 10.4 μg/mg | 666.7 μg/ml |
| | (24-fold dilution) | (192-fold dilution) | (3-fold dilution) |
| 6 | 83.3 μg/mg | 125 μg/ml | 666.7 μg/ml |
| | (24-fold dilution) | (16-fold dilution) | (3-fold dilution) |

It is worth noting that cell damage was not caused even when the concentration of the sample stock solution was higher than the one in the previous test. In the experiments shown in Table 5, the 1:24 dilution indicates that it was not effective in No. 6 test tube but it was effective in the 1:24 dilution which is lower than in No. 5 test tube (1:16 dilution), and this was abbreviated as 5-6To. Likewise, the 1:192 dilution indicates that it was not effective in No.9 test tube (1:256 dilution), but it was effective in the 1:129 dilution which is lower than in No. 8 test tube No.8, and this was abbreviated as 8-9To. The 1:3 dilution indicates that it was not effective in sample 3 (1:4 dilution) but it was effective in the dilution between sample 3 and sample 2 (1:2 dilution), and this was abbreviated as 2-3 To. As a result of the findings, sample 5 and sample 6 were proven to have strong anti-influenza virus activity.

6. Antibacterial Activity Test

Next an evaluation test for antibacterial activity was run for 4 species of guaiacol (sample 1), lignin sulfonic acid (sample 2),2,6-dimethoxyphenol (sample 3) and 3,5 dimethoxyphenol (sample 4) selected among chemical substances in the present invention, according to the following procedure (other samples 5, 6, 11-14 is described hereunder).

A Test Procedure

Agar plate is prepared as a medium for sensitivity disks. The plate is inoculated with 25 ml of one of the six test microorganism suspensions (prepared to 10⁶/ml), as shown in Table 6, and after cultured for 48 hr at 37° C., it is subsequently checked for the growth of the organism concerned. The result is presented in the following Table 6, in which (−) means absence of growth, and (+) indicates positive.

TABLE 6
Results of antimicrobial activity test (1)

| | number | sample | sample | sample | sample |
|---|---|---|---|---|---|
| strain | (ml) | 1 | 2 | 3 | 4 |
| E. coli O157 | 2.4 × 10⁶ | (−) | (−) | (−) | (−) |
| K. pneumoniae | 3.1 × 10⁶ | (−) | (−) | (−) | (−) |
| S. Enteritidis | 1.9 × 10⁶ | (−) | (−) | (−) | (−) |
| P. aerginos | 4.2 × 10⁶ | (−) | (−) | (−) | (−) |
| S. aureus | 2.7 × 10⁶ | (−) | (−) | (−) | (−) |
| B. subtilis | 6.1 × 10⁵ | (−) | (−) | (−) | (−) |

As shown in Table 6, guaiacol (sample 1), lignin sulfonic acid (sample 2),2,6-dimethoxyphenol (sample 3) and 3,5-dimethoxyphenol (sample 4) were proved to have the growth inhibitory effects against all the strains. Through the antimicrobial activity test, similar to the one described above, the said guaiac resin also appeared to have the equal or superior growth inhibitory effects to guaiacol.

Next, according to a different procedure, an evaluation test using 6 types of bacteria was performed on the growth inhibitory effects of the remaining samples, which are lignosulfonic acid sodium salt (sample 5) lignosulfonic acid sodium salt acetate (sample 6), lignin organosolv propionate (sample 11), 4-benzyloxyguaiacylglycerol-β-guaiacylether (sample 12), syringaldehyde (sample 13)

B. Test Procedure

Aqueous solutions of each specimen (powders) at a concentration of 8% was diluted 1:40 as the specimen stock solution for MIC measurement. The stock solution diluted at a graduation ratio of 1:40 to 1:400 and the stock solution were added to the agar culture medium on which 6 test viruses were inoculated, and incubated at 30° C. for 17 hours. Growth inhibition bands were examined on the agar culture medium. The results of the experiment are presented in Table 7. The specimen stock solution was prepared by diluting 25 ml of 8% solution with 1000 cc of distilled water(1:40 dilution), which is converted into the amount per 2 mg/ml by solid content.

TABLE 7
Results of antibacterial activity test (2)

| | sample | | | | | |
|---|---|---|---|---|---|---|
| strain | 5 | 6 | 11 | 12 | 13 | 14 |
| E. coli O157 | <40-fold | <40-fold | <40-fold | <40-fold | <40-fold | <40-fold |
| K. pneumoniae | <40-fold | <40-fold | <40-fold | <40-fold | <40-fold | <40-fold |
| S. Enteritidis | <40-fold | <40-fold | <40-fold | <40-fold | <40 fold | <40-fold |
| P. aerginos | <40-fold | <40-fold | <40-fold | <40-fold | <40-fold | <40-fold |
| S. aureus | <40-fold | <40-fold | 40-fold | <40-fold | <40-fold | <40-fold |
| B. subtilis | <40-fold | <40-fold | <40-fold | <40-fold | <40-fold | <40-fold |

In Table 7, “<40-fold” indicates that the complete growth inhibition was not observed in the 1:40 dilution and the concentration ratios should be not more than 1:40 dilution (ex. 30-fold=3 mg/ml, 20-fold=4 mg/ml). The sample that exhibited complete inhibition against S. aureus in the 1:40 dilution (2 mg/ml) was lignin organosolv (sample 11). However, the remaining samples that were indicative of “<40-fold (lower than the 1:40 dilution)” were proven to have a certain degree of inhibitory effects. They are expected to show the complete inhibitory effects against the bacterial growth, by increasing the concentration level. As a consequence, the samples, 5, 6, 11-14 were recognized to have sufficient useful antibacterial activity.

7. Examples

Among many antimicrobial agents of the present invention, lignosulfonic acid sodium salt and lignosulfonic acid sodium salt acetate were selected and taken orally by persons who infected with influenza virus. Details about the effects on them are described below.

A. Case Study 1: a 51 Year-Old Male, March, 2000

A 51 year-old male got influenza, while taking a nap in a room with low temperature and low humidity and. He had been tired from working till late at night over three months. At first, he began to suffer from coughing spells, which increased to 60 times in 30 minutes. While sleeping, he also perspired. Even if taking a sauna, he felt a chill at the back of his body. Because he had a bad cough, he took 1 g of lignosulfonic acid sodium salt acetate dissolved in 200 cc of lukewarm water. Immediately after taking it, he felt that the cough reflex was suppressed. The coughing spells decreased sharply to 10 times in 30 minutes. He continued to take the agent four times daily as a rough standard, whereby a sore throat and pain around lungs through coughing fits decreased. Under ordinary conditions, he would not have resumed his work for seven days. However, this time he only took two days off work. He recovered from influenza, while working at usual workplace. After recovery, the slight cough persisted for a short time. During the disease, he conserved his strength. Lignosulfonic acid sodium salt acetate showed that it had a beneficial effect on the influenza virus, preventing loss of bodily strength caused by the virus, decreasing the cough reflex without symptoms like loss of appetite and so on. Side effects were not seen.

B. Case Study 2: a 54 Year-Old Female, March, 2001

The patient is of a type most likely to contract influenza. During this season every year, he contracted it several times without fail. Her symptoms frequently included low blood pressure, loss of appetite and severe inflammation of her bronchial tube. For seven to ten days she had a feeling of such lassitude that she had to go to the lavatory by crawling along the floor. While she was working in March 2001, early symptoms such as a sense of discomfort in the throat and a cough appeared. In previous years, her condition worsened after 2 days and she would have fallen down due to low blood pressure. She would repeat the same routine, to come and take complete rest after being given an intravenous drip injection at the hospital. Therefore, she gargled with 200 cc of hot water dissolved 1 g of lignosulfonic acid sodium salt. After that, a sense of discomfort in the throat disappeared and her voice returned to normal. The sickness symptoms were suppressed and side effects were not seen. For persons who are very weak against influenza virus, lignosulfonic acid sodium salt disclosed in the invention was effective.

8. Results of the Invention

All the chemical substances that have been confirmed to have the said antiviral and antibacterial activities were used in solitary application in the present invention. However, as mentioned in the description of the combined use of 3,5-dimethoxyphenol and other agents, the said samples 1-13 may be used in solitary application as well as in combined application. Two or more samples may be combined, or one or more samples may be coupled with other substances which were not disclosed in the present invention according to need, whereby antiviral and antimicrobial activity shown for each substance are not diminished but enhanced, decreasing cell damage.

The substances which have demonstrated remarkable antiviral activity and antibacterial activity in the present invention are widely available for drugs such as anti-viral agents, anti-bacterial agents and antimicrobial agents. It can also be used as health food ingredients having multi-purpose effects. Needless to say, the samples such as lignosulfonic acid sodium salt (sample 5) and lignosulfonic acid sodium salt acetate (sample 6) that cell damage was not seen in the cell-level tests were confirmed to be safe for oral administration. (Non-toxicity of them were certified by the manufacturers.) Substances with relatively strong cytopathy, 2,6-dimethoxyphenol (sample 3) for example, can be treated as safe substances according to conventional observations. According to the literature which is broadly acknowledged, 2,6-dimethoxyphenol as well as guaiacol, syringaldehyde, etc. have been proven to have a high level of safety, on the ground that LD₅₀ by intraperitoneal administration and oral administration in mice is more than 1,000 mg/kg. And it is also stated that a conventional-dose of guaiacol by oral administration and external application is 0.2 g/once (0.0.6 g/daily) and the toxicity level in a human(ORL-HMN) is determined to be 43 mg/kg. Accordingly, from today's level of technology, it can be said that there is no problems with safety on all the substance which concerned with the present invention.

As described in detail above, in the present invention, candidate substances were selected from many chemical substances produced as industrial products and they were evaluated extensively. Antiviral activities (against AIDS and influenza virus) and antibacterial activities are confirmed from various angles regarding the said 13 types of chemical substances disclosed in the present invention. These were selected as the substances that exert not only unexpected positive effects for specific targets such as viruses and bacteria independently but also exert multi-purpose effects from antiviral activity to antibacterial activity. As a result of the findings they attained a new position as antimicrobial agents. This may provide effective and relatively inexpensive antimicrobial agents, which may contribute to the improvement of mankind's health and welfare. The new agents will have a practical effect on society providing a stable and continuous supply, through using them on a daily basis, either given alone or in combination with food and drink.

What is claimed is:

1. Antimicrobial agents comprising one or more active constituents having antimicrobial activity, which are selected from guaiacol, lignin sulfonic acid, 2,6-dimethoxyphenol, 3,5-dimethoxyphenol, lignosulfonic acid sodium salt, lignosulfonic acid sodium salt acetate, lignin organosolv propionate, 4-benzyloxyguaiacylglycerol-β-guaiacylether and syringaldehyde.

2. Antimicrobial agents as claimed in claim 1, wherein the said active constituent is one type selected from guaiacol, lignin sulfonic acid, 2,6-dimethoxyphenol, 3,5-dimethoxyphenol, lignosulfonic acid sodium salt, lignosulfonic acid sodium salt acetate, lignin organosolv propionate, 4-benzyloxyguaiacylglycerol-β-guaiacylether and syringaldehyde, and the said antimicrobial activities are antiviral activity and antibacterial activity.

3. Antimicrobial agents as claimed in claim 1, wherein the said active constituents are two or more types selected from guaiacol, lignin sulfonic acid, 2,6-dimethoxyphenol, 3,5-dimethoxyphenol, lignosulfonic acid sodium salt, lignosulfonic acid sodium salt acetate, lignin organosolv propionate, 4-benzyloxyguaiacylglycerol-β-guaiacylether and syringaldehyde, and the said antimicrobial activities are antiviral activity and antibacterial activity.

4. Antimicrobial agents as claimed in claim 2 and 3, wherein the said antiviral activities is concerned with anti-AIDS virus activity and anti-influenza virus activity, and the said antibacterial activity is antibacterial activity against E.coli O157, K.pneumoniae, S.Enteritidis, P.aerginos, S.aureus and B.subtilis.

5. Antimicrobial agents as claimed in claim 2, wherein the said active constituents are lignosulfonic acid sodium salt or lignosulfonic acid sodium salt acetate, particularly salient in anti-AIDS virus activity.

6. Antimicrobial agents as claimed in claim 2, wherein the said active constituent is lignin organosol propionate and is excellent in antibacterial activity.

7. Antimicrobial agents as claimed in claim 2, wherein the said active constituent is syringaldehyde and the said antimicrobial activities are particularly anti-AIDS virus activity and anti-influenza virus activity.

8. Antimicrobial agents as claimed in claim 2, wherein the said active constituent is guaiac resin including guaiacol and the said antimicrobial activity is antiviral activity.
